(19) United States
(12) Patent Application Publication
(10) Pub. No.: US 2013/0116517 A1
(43) Pub. Date: May 9, 2013

(54) METHOD AND MEASURING DEVICE FOR GATHERING SIGNALS MEASURED IN VITAL TISSUE

(71) Applicants: Holger Jungmann, Gelsenkirchen (DE); Michael Schietzel, Herdecke (DE)

(72) Inventors: Holger Jungmann, Gelsenkirchen (DE); Michael Schietzel, Herdecke (DE)

(21) Appl. No.: 13/639,345

(22) PCT Filed: Apr. 11, 2011
(86) PCT No.: PCT/EP11/01789

§ 371 Date: Jan. 16, 2013

(30) Foreign Application Priority Data
Apr. 9, 2010 (DE) 10 2010 014 593.9

(51) Int. Cl. A61B 5/1495; A61B 5/1455

(52) U.S. Cl. CPC A61B 5/1495; A61B 5/1455
USPC 600/310

(57) ABSTRACT

The invention relates to a method for calibrating a spectrometer equipped with a CCD array, the CCD array recording a spectrum from a reference volume emitter. The raw data hereby recorded are used to generate a function which describes an etaloning effect that occurs, said function being saved in the spectrometer as a correction function for measurements obtained from volume emitters.

The invention concerns a method and a measuring instrument for collecting test signals from living tissue, especially for determining the composition of body liquids as well as of maybe only temporarily vascular-bound substances.

Measurement methods are known, in which an analysis of temporarily vascular-bound substances is done by applying a mobile spectrometer to a corresponding tissue area and recording, by this movable spectrometer, the spectrum of reflected light emerging from the tissue. By means of the spectrum recorded in this way various substances present in the examined tissue area can be detected. These spectrometers can be structured as classic spectrometers, in which the incident light is split by optical means and the intensity of the split light is measured by associating it to the wavelength. For avoiding movable parts the spectrometers can be formed in such a way that the light split according to its wavelength is led onto a CCD array and is analyzed by it.

The object of the invention is to create solutions, by which by means of a spectrometric measurement using CCD arrays measured values can be generated that distinguish themselves by a particularly high representativity.

This task is solved according to the invention by a process for the calibration of a spectrometer equipped with a CCD array in which the CCD array records a first calibration spectrum and a second calibration spectrum, in which for generating these calibration spectra reference structures are illuminated that distinguish significantly as for the escape depth of the light emitted by them during the reference measurement.

It is thus advantageously possible to determine a correction system by which the recording characteristic of the CCD arrays used in each case be described and standardized within the device.

This correction system can be deposited for example as characteristic field or parameterized correction function in a command unit of the spectrometer.

According to a particularly preferred embodiment of the invention several correction systems for certain substances are generated, so that for example for the measurement of selected tissue or blood components each time optimized correction systems can be used.

Preferably one of the samples is a surface emitter, and the other sample is a volume emitter. These reference emitters can be formed in such a way that they irradiate a substantially white light.

It is possible to carry out the measurement in such a way that by it in an evaluation step depth information for the origin depth of the recorded light is obtained. On the basis of this depth information if need be the correction system can be further refined. The depth information can be obtained especially by taking into account, and according data processing, of signal changes caused by opacity.

Preferably several correction systems are generated by using several master samples in which a substance reference is contained in different concentrations. For each substance preferably a master sample is provided that guarantees a light emission without deep penetration of the illumination light. A master sample preferably containing the same substance can be formed in such a way that this substance is embedded in a translucent base. The translucent base can be formed in such a way that it, as for its opacity characteristics, corresponds to the opacity characteristics of an opacity characteristic that is typical for the body position to be examined spectrometrically.

The calibration according to the invention of the spectrometer can take place advantageously by leading it over several master samples that distinguish as for the origin depth of the emitted light. The spectra obtained in this way can be used for generating the correction system by an electronic signal processing device integrated directly into the spectrometer. Preferably however the obtained spectra are selected by an interface device and led to a separate computer system. Over this computer system then a correction function can be generated that in a following procedural step is deposited in the evaluation device of the spectrometer.

It is also possible to deposit the correction function in a computer for example accessible via Internet, associated with an identification code of the spectrometer. This special correction function can then be accessed selectively by the user of the spectrometer or by a user entrusted with the evaluation of the spectra.

It is also possible, to realize the method according to the invention in such a way that a calibration model can be requested by the user of the spectrometer, which allows to obtain two spectra from significantly different reflection depths. The spectra obtained by the user can be compared then using a master spectrum obtained from other sources for this calibration standard. On the basis of that comparison a calibration of the spectrometer or a normalization of the measured values can be done.

Further particulars and characteristics of the invention result from the following description in connection with the drawing. The figures show:

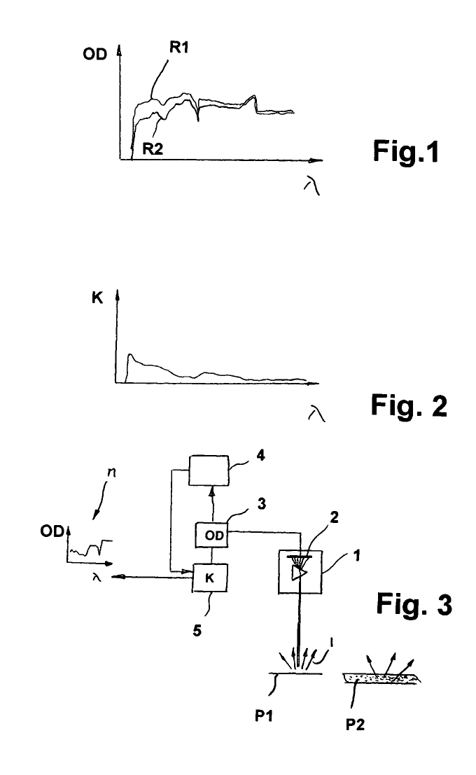

FIG. 1 a sketch to illustrate the variations of the optic density, or intensity of spectral components of identical substances during the resolution of irradiated light from two calibration samples, which are configured in such a way that they condition significantly different light irradiation depths;

FIG. 2 a sketch to illustrate a correction function generated from the double reference measurement according to FIG. 1;

FIG. 3 a schematic representation to illustrate the use of the correction function according to the invention for providing standardized measured values.

FIG. 1 shows two spectra obtained using a spectrometer that includes a CCD array. The spectra were obtained from two samples (P1 and P2 in FIG. 3). These samples act as calibration standards and are designed in such a way that this one reference substance in the calibration standard is adapted in such a way that for one of the calibration standard an irradiation extremely near to the surface of the light to be examined results, whereas the other calibration standard is configured in such a way that the light to be examined is irradiated from deeper and again different depths.

The difference of both these spectra allows to quantify a systematic signal recording effect conditioned by the CCD array, especially by an oxide layer of the CCD array, and basing on this, to adapt a calibration or normalization system.

This normalization system can be represented as a characteristic field, or, as shown typically in FIG. 2, as a correction function.

This correction function can be deposited in the spectrometer, so that this directly outputs accordingly standardized measurement results.
The correction function can also otherwise be considered subsequently, for example for special post processing, when for example measurement results determined by different equipment are to be related to each other.

As shown in FIG. 3, spectra of samples P1 and P2 are recorded, which samples are configured in such a way that the light each time coupled into the spectrometer L is irradiated once almost completely from an area extremely near to the surface, and in case of the sample P2 from deeper, preferably also diverging depths. The light accordingly collected is led to a spectrometer 1. The spectrometer comprises a CCD array 2. The signals detected by the CCD array 2 are deposited in a first storage 3 for example as raw values of the optic density OD. These raw values are read by a calibration computer 4. The calibration computer 4 generates a calibration function K on the basis of the spectra measured at least for the two special samples P1 and P2 (cf FIG. 2). This calibration function K is deposited in a signal processing device 5 of the measuring device. The measurement results M made available to a user in the end for later measurements are standardized taking into account this calibration function in the signal processing device 5.

One of the samples P1 and P2 constitutes a volume emitter. This sample is preferably configured in such a way that it causes an opacity typical for vital tissue.

The calibration function K can be formed in such a way that it also describes dynamic characteristics occurring in the spectra. When measuring from diffuse depths, for narrow wavelength ranges each time a dynamic value can be established, on the basis of which a representative value of the optic density is determined. The correction function describes in the form of a derivation a variance of the raw data called etaloning.

1. A process for the calibration of a spectrometer equipped with a CCD array, in which the CCD array records a first calibration spectrum and a second calibration spectrum, in which for generating these calibration spectra reference structures are illuminated, that distinguish significantly as for the escape depth of the light emitted by them.

2. The process according to claim 1, wherein from these two calibration spectra a correction system is determined, by which the recording signals of the CCD array used in each case are individually standardized.

3. The process according to claim 2, wherein the correction system is deposited as characteristic field or parameterized correction function in a command unit of a corresponding spectrometer.

4. The process according to claim 3, wherein several correction systems for certain substances are generated.

5. The process according to claim 1, wherein for example for the measurement of selected tissue or blood components each time optimized correction systems are used.

6. The process according to claim 1, wherein from the latter in an evaluation step depth information for the origin depth of the recorded light is obtained.

7. The process according to claim 1, wherein on the basis of this depth information the correction system is further refined.

8. The process according to claim 1, wherein several correction systems are generated by using several master samples in which a substance reference is contained in different concentrations.

9. A process for the calibration of a spectrometer equipped with a CCD array in which the CCD array records a spectrum from a reference volume emitter, in which based on the recorded raw data a function is generated that describes an etaloning effect arising here, and in which this function is deposited in the spectrometer as a correction function for measurements from volume emitters.
